(19) United States
(12) Patent Application Publication
(10) Pub. No.: US 2024/0018074 A1
(43) Pub. Date: Jan. 18, 2024

(54) METHOD FOR REDUCING CHLOROFLUOROCARBON IMPURITIES IN THE MANUFACTURE OF TRANS-1,3,3,3-TETRAFLUOROPROPENE (HFO-1234ze(E))

(71) Applicant: Honeywell International Inc., Charlotte, NC (US)

(72) Inventors: Richard D. Horwath, Richmond, VA (US); Haluk Kopkalli, Staten Island, NY (US); Gustavo Cerri, Parsippany, NJ (US); Joshua Close, Cheektowaga, NY (US); Daniel C. Merkel, Orchard Park, NY (US); Jennifer W. McClaine, Branchburg, NJ (US); Alex C. Fattore, Charlotte, NC (US); Hang T. Pham, Amherst, NY (US); Carlos Navar, Baton Rouge, LA (US); Justin Howard, Lakeview, NY (US)

(21) Appl. No.: 18/218,691

(22) Filed: Jul. 6, 2023

Related U.S. Application Data
(60) Provisional application No. 63/389,174, filed on Jul. 14, 2022.
(60) Provisional application No. 63/415,457, filed on Oct. 12, 2022.

(51) Int. Cl. C07C 17/383; C07C 17/20; B01D 3/14

(52) U.S. Cl. CPC C07C 17/383; C07C 17/206; B01D 3/143

(57) ABSTRACT

Two methods for reducing CFC impurities and, in particular, CFC-114, in the HFO-1234ze(E) production process from the reaction of CFC-113 and HF. The first method involves subjecting an intermediate or recycle stream to separation and distillation to purge CFC-113 from the process. The second method involves operating the separation at a higher pressure avoids the formation of an azeotrope between CFC-113 and HFC-245fa. The CFC-113/HFC245fa azeotrope is discussed, as well as other optional processes for removal or mitigation of CFC-113 which include further separations that remove CFC-114 from the HFO-1234ze(E) product and/or CFC-113 from the HFC-245fa feed to produce an HFO-1234ze(E) product that is largely free from CFC-114 and other CFC impurities.

### CROSS-REFERENCE TO RELATED APPLICATION

This application claims priority to U.S. Provisional Application No. 63/389,174, filed Jul. 14, 2022, and U.S. Provisional Application No. 63/415,457, filed Oct. 12, 2022, both of which are herein incorporated by reference in their entireties.

### FIELD

The present disclosure is directed to a method for reducing the amount of chlorofluorocarbon impurities in the process for producing trans-1,3,3,3-tetrafluoropropene (HFO-1234ze(E)).

### BACKGROUND

Chlorofluorocarbons (CFCs) have found widespread use in a number of applications, including as refrigerants, aerosol propellants, blowing agents, heat transfer media, gaseous dielectrics, and fire suppression. In recent years, there has been widespread concern that certain chlorofluorocarbons might be detrimental to the Earth's ozone layer. As a result, there is a worldwide effort to use halocarbons which contain fewer or no chlorine substituents. Accordingly, the production of hydrofluorocarbons, or compounds containing only carbon, hydrogen and fluorine, has been the subject of increasing interest to provide environmentally desirable products for use as solvents, blowing agents, refrigerants, cleaning agents, aerosol propellants, heat transfer media, dielectrics, fire extinguishing compositions, power cycle working fluids, and starting material for producing hydrofluoroolefins (HFOs) that do not harm the ozone layer and also have low global warming potential.

### SUMMARY

The present disclosure is based on the discovery that CFC impurities and, in particular, CFC-114, forms in the HFO-1234ze(E) production process from the reaction of CFC-113 with HF. It has been found that it is possible to reduce the amount of such CFC impurities by subjecting an intermediate or recycle stream to separation and distillation to purge CFC-113 from the process. It has also been found that it is possible to reduce the amount of CFC impurities by operating the separation at higher pressures to avoid an azeotrope that forms between CFC-113 and HFC-245fa. Other optional processes for removal or mitigation of CFC-113 include further separations that remove CFC-114 from the HFO-1234ze(E) product and/or CFC-113 from the HFC-245fa feed to produce an HFO-1234ze(E) product that is largely free from CFC-114 and other CFC impurities.

In one form thereof, the present disclosure provides a process for producing HFO-1234ze(E), including: reacting a feed stream comprising HFC-245fa and CFC-113 in a reactor in the presence of a catalyst to form a first product stream comprising HFO-1234ze(E), unreacted HFC-245fa, and CFC-113; separating the first product stream into a second product stream containing HFO-1234ze(E) and a third product stream containing unreacted HFC-245fa and CFC-113; distilling the third product stream to produce an overhead recycle stream and a bottoms stream, the recycle stream containing unreacted HFC-245fa and a first amount of CFC-113, and the bottoms stream containing a second amount CFC-113 greater than the first amount; and conveying the recycle stream back to the feed stream.

In a further form thereof, the present disclosure provides a process for removing CFC-113 from the HFC-245fa feed stream in the production of HFO-1234ze(E) including: feeding HFC-245fa to a separation device, the HFC-245fa further including a first amount of CFC-113; recovering, from the separation device, a purified HFC-245fa stream having a second amount of CFC-113 less than the first amount.

In a further form thereof, the present disclosure provides a process for removing CFC-114 from the HFO-1234ze(E) product stream in the HFO-1234ze(E) process including feeding HFO-1234ze(E) to a separation device, the HFO-1234ze(E) further including a first amount of CFC-114; recovering, from the separation device, a purified HFO-1234ze(E) stream having a second amount of CFC-114 less than the first amount.

In a further form thereof, the present disclosure provides an azeotrope or azeotrope-like composition consisting of CFC-113 and HFC-245fa.

### BRIEF DESCRIPTION OF THE DRAWINGS

The above mentioned and other features of the disclosure, and the manner of attaining them, will become more apparent and the disclosure itself will be better understood by reference to the following description of embodiments of the disclosure taken in conjunction with the accompanying drawings.

FIG. 1 provides a flow diagram for the process to remove CFC-113 and CFC-114 in the HFO-1234ze(E) process.

FIG. 2 is a plot of temperature vs. weight percent HFC-245fa measured in an ebulliometer study for HFC-245fa and CFC-113 according to Example 1.

FIG. 3 is a plot of temperature vs. composition of HFC-245fa (mass %, relative to CFC-113) at a pressure of 5 psia.

FIG. 4 is a plot of temperature vs. composition of HFC-245fa (mass %, relative to CFC-113) at a pressure of 10 psia.

FIG. 5 is a plot of temperature vs. composition of HFC-245fa (mass %, relative to CFC-113) at a pressure of 14.7 psia.

FIG. 6 is a plot of pressure vs. the azeotrope composition of HFC-245fa and CFC-113 (mass %).

The exemplification set out herein illustrates an embodiment of the disclosure, and such exemplification is not to be construed as limiting the scope of the disclosure in any manner.

### DETAILED DESCRIPTION

### I. Definitions

TABLE 1
Names of compounds as used herein
and corresponding IUPAC names

| Name | IUPAC Name |
|---|---|
| CFC-113 | 1,1,2-trichloro-1,2,2-trifluoroethane |
| CFC-113a | 1,1,1-trichloro-2,2,2-trifluoroethane |
| CFC-114 | 1,2-dichloro-1,1,2,2-tetrafluoroethane |
| CFC-114a | 1,1-dichloro-1,2,2,2-tetrafluoroethane |
| HFC-245fa | 1,1,1,3,3-pentafluoropropane |
| HFO-1234ze(E) | trans-1,3,3,3-tetrafluoropropene |
| HFO-1234ze(Z) | cis-1,3,3,3-tetrafluoropropene |

As used herein, the terms “CFC-113” and “CFC-114” refer to both CFC-113 and CFC-113a and CFC-114 and CFC-114a respectively.

Unless otherwise specified, the term “HFO-1234ze” refers to both HFO-1234ze(E) and HFO-1234ze(Z) together.

### II. Methods for Reducing CFC-114 in HFO-1234ze(E)

The present disclosure is based on the discovery that CFC-114 and CFC-114a form in the HFO-1234ze(E) production process from the reaction of CFC-113 and CFC-113a, which are present in the HFC-245fa feed, with HF that is produced by the dehydrofluorination reaction of HFC-245fa. These CFCs are impurities, as well as ozone depleting substances and therefore it is desired to control the content of CFCs in the HFO-1234ze(E) product. In some jurisdictions, regulations limit the level of these impurities for certain emissive uses.

Therefore, methods to remove CFC-113 from the HFC-245fa feed and CFC-114 from the HFO-1234ze(E) final product are needed. This disclosure is intended to address the removal of CFC-113 and CFC-114 at several locations or unit operations during the HFO-1234ze(E) manufacturing process.

HFO1234ze(E) may be produced industrially by the dehydrofluorination reaction shown below in Equation 1.

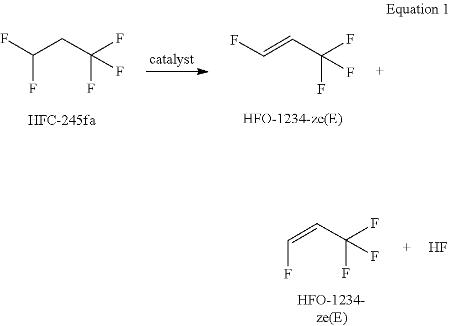

The production of HFO-1234ze(E) involves the catalytic conversion of HFC-245fa by dehydrofluorinating HFC-245fa to produce a mixture comprising a combination of cis- and trans-isomers of HFO-1234ze and hydrogen fluoride. Preferably, dehydrofluorination of HFC-245fa may be carried out in the vapor phase such as in a fixed-bed reactor. The dehydrofluorination reaction may be conducted in any suitable reaction vessel or reactor, but it should preferably be constructed from materials that are resistant to the corrosive effects of hydrogen fluoride such as nickel and its alloys including Hastelloy, Inconel, Incoloy, and Monel or vessels lined with fluoropolymers.

The conversion of HFC-245fa to HFO-1234ze(E) and HFO-1234ze(Z) is limited by equilibrium. Accordingly, the reactor product stream is often passed through several separation steps to recover HFO-1234ze(E) as a product and to recycle unconverted HFC-245fa to the reactor.

If the feed HFC-245fa stream contains impurities such as CFC-113, the impurities will build up in the recycle stream which may hinder the conversion and yield of the dehydrofluorination reaction. For example, if the recycle stream comprises CFC-113, a portion of the CFC-113 entering the reactor may react with the HF produced by the HFC-245fa dehydrofluorination reaction, converting CFC-113 to CFC-114 by the reaction shown below in Equation 2.

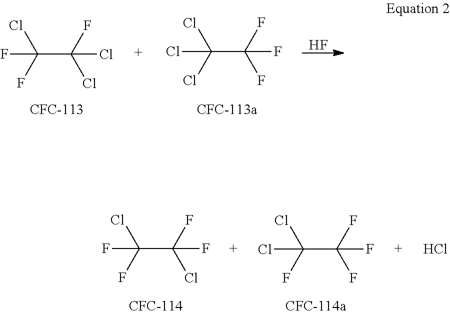

The formation of CFC-114 in the reactor is a significant drawback because it is an undesirable impurity and difficult to separate from HFO-1234ze(E), particularly at low CFC-114 concentrations. Another alternative to reduce the buildup of CFC-113 and CFC-114 would involve distilling the feed HFC-245fa stream to remove CFC-113. However, it has been found that separation of CFC-113 from HFC-245fa is difficult, particularly at low concentrations of CFC-113 where, as discussed below, a CFC-113/HFO-245fa azeotrope may be present and, as also discussed further below, such azeotrope is prevalent particularly at low pressures.

### III. Process for Producing HFO-1234ze(E)

A general process for producing HFO-1234ze(E) is described below. The present disclosure encompasses two methods which may be incorporated into the HFO-1234ze(E) process to reduce the level of CFC impurities in the HFO-1234ze(E) product.

In the first method, discussed in more detail in section IV, CFC-113 may be purged from an intermediate stream in the process where the separation is easier so that smaller, less expensive equipment can be used with lower energy consumption and lower yield losses. Subjecting this intermediate stream to a separation and distillation provides a recycle stream comprising unconverted HFC-245fa, HFO-1234ze(Z), HFO-1234ze(E), and reduced amounts of CFC-113 which can be fed back into the reactor, and a waste stream comprising impurities which can be removed from the system.

In the second method, discussed in more detail in section V, the reaction mixture is exposed to an increased pressure during the separation stage which avoids the formation of a homogenous minimum boiling point azeotrope at about 3.5 wt. % CFC-113 and about 96.5 wt. % HFC-245fa and at a temperature of about 14.44° C.±0.3° C. and a pressure of about 14.29 psia±0.3 psia. This azeotrope is difficult to separate and is discussed in more detail in section VI and Example 1. Avoiding the formation of an azeotrope or azeotrope-like mixture of CFC-113 and HFC-245fa may decrease yield losses by reducing excess purging of HFC-245fa.

To reduce the level of CFC impurities further, either system may optionally be combined with several auxiliary separation devices to reduce the amount of CFC-113 in the HFC-245fa feed, (prior to the intermediate distillation) and to remove any residual CFC-114 from the HFO-1234ze(E) product.

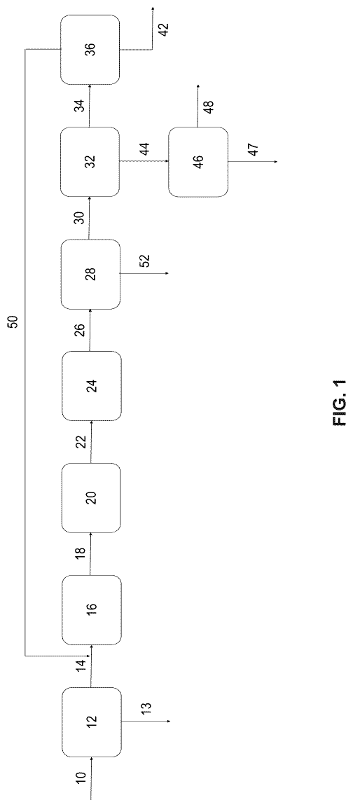

An example of the general HFO-1234ze(E) process is shown in FIG. 1 and summarized below. As shown therein, crude HFC-245fa enters the process via feed stream 10. Stream 10 is optionally passed through separation device 12 which initially reduces the level of CFC-113 in the HFC-245fa feed via one of several unit operations described below.

The composition in feed stream 10 may comprise HFC-245fa raw material and CFC-113 present as an impurity. Feed stream 10 may comprise CFC-113 in an amount greater than 0 ppm such as 1 ppm or more, or an amount as little as 5000 ppm or less, 4500 ppm or less, 4000 ppm or less, 3500 ppm or less 3000 ppm or less, 2500 ppm or less, 2000 ppm or less, 1500 ppm or less, 1000 ppm or less, 900 ppm or less, 800 ppm or less, 700 ppm or less, 600 ppm or less, 500 ppm or less, 400 ppm or less, 300 ppm or less, 200 ppm or less, 100 ppm or less, 50 ppm or less, 20 ppm or less, 10 ppm or less or within any range encompassing any two of these values as endpoints.

Separation device 12 may be any separation device suitable for decreasing the level of CFC-113 in the HFC-245fa. In one embodiment, the separation devices may comprise a zeolite that exploits structural or size differences between the molecules. Suitable zeolites include sodium silicoaluminate, zeolite type X, AW-500, 3 A molecular sieves, 4 A molecular sieves, 5 A molecular sieves, activated carbon, or carbon molecular sieves. In one embodiment, the separation devices may comprise a zeolite that exploits the difference in dipole moment between the molecules. In one embodiment, the separation device may comprise an extractant to exploit the different solubility between the molecules. Suitable extractants include but are not limited to pentane, hexane, ethyl acetate, dichloromethane, chloroform, tetrahydrofuran, methanol, or water. In one embodiment, the separation device may comprise an azeotropic distillation apparatus. In one embodiment, the separation device may comprise an apparatus to pass the mixtures in vapor phase through mineral oil at a velocity low enough to effect dissolution of undesired CFC impurities in mineral oil and a knockout pot to eliminate oil from the desired component.

Stream 13 comprises CFC-113 which may be removed from the system for disposal.

Stream 14, also referred to herein as the first product stream, conveys purified HFC-245fa into reactor 16, where HFC-245fa is catalytically dehydrohalogenated to produce a product comprising HFO-1234ze(E) as described above.

The composition in stream 14 may comprise CFC-113 in an amount greater than 0 ppm such as 1 ppm or more, or an amount as little as 5000 ppm or less, 4500 ppm or less, 4000 ppm or less, 3500 ppm or less 3000 ppm or less, 2500 ppm or less, 2000 ppm or less, 1500 ppm or less, 1000 ppm or less, 900 ppm or less, 800 ppm or less, 700 ppm or less, 600 ppm or less, 500 ppm or less, 400 ppm or less, 300 ppm or less, 200 ppm or less, 100 ppm or less, 50 ppm or less, 20 ppm or less, or 10 ppm or less, above 0 ppm or 0 ppm or within any range encompassing any two of these values as endpoints.

The reactor 16 may be operated at a temperature as low as 300° F., 325° F., 350° F., 375° F., 400° F., 425° F., 450° F., 475° F., 500° F., 525° F., 550° F., or as high as 575° F., 600° F., 625° F., 650° F., 675° F., 700° F., 725° F., 750° F., 775° F., 800° F., or within any range encompassing any two of these values as endpoints. The reactor 16 may also be operated at a pressure as low as 0 psia, 5 psia, 10 psia, 15 psia, 20 psia, 25 psia, 30 psia, 35 psia, 40 psia, 45 psia, 50 psia, or as high as 55 psia, 60 psia, 65 psia, 70 psia, 75 psia, 80 psia, 85 psia, 90 psia, 95 psia, 100 psia, or within any range encompassing any two of these values as endpoints. The residence time in reactor 16 may be as low as 0 seconds, 5 seconds, 10 seconds, 15 seconds, 20 seconds, 25 seconds, 30 seconds, 35 seconds, 40 seconds, 45 seconds, 50 seconds, or as high as 55 seconds, 60 seconds, 65 seconds, 70 seconds, 75 seconds, 80 seconds, 85 seconds, 90 seconds, 95 seconds, 100 seconds, or within any range encompassing any two of these values as endpoints.

Dehydrofluorination catalysts may include chromium oxides, chromium oxyfluorides, and chromium halides. The chromium oxides may include amorphous chromium oxide (Cr₂O₃), crystalline chromium oxide, and combinations of the foregoing. The chromium oxyfluorides may include fresh amorphous chromium oxide (Cr₂O₃) pretreated with HF, fresh crystalline chromium oxide (Cr₂O₃) pretreated with HF, amorphous chromium oxyfluoride (CrOₓF_{y}, where x may be greater than 0 but less than 1.5, and y may be greater than 0 but less than 3), crystalline chromium oxyfluoride (CrOₓF_{y}, where x may be greater than 0 but less than 1.5, and y may be greater than 0 but less than 3), and combinations of the foregoing. In one embodiment, the catalyst is amorphous chromium oxyfluoride (CrOₓF_{y}, where x may be greater than 0 but less than 1.5, and y may be greater than 0 but less than 3). The chromium halides may include chromium trifluoride (CrF₃), chromium trichloride (CrCl₃), chromium triiodide (CrI₃) and chromium tribromide (CrBr3), and combinations of the foregoing. In one embodiment, the catalyst is chromium trifluoride (CrF₃).

Other suitable catalysts include promoted or doped, also referred to as modified, chromium-based catalysts, which are based on chromium and include an amount of at least one co-catalyst or modifier selected from K, Na, Cu, Ni, Zn, Co, Mn, Mg, or mixtures thereof. The amount of the co-catalyst or modifier may be between 0.1 wt. % and 20 wt. % based on the total weight of the catalyst and, more particularly, may be present in an amount as little as 0.1 wt. %, 0.5 wt. %, 1.0 wt. % 1.5 wt. % or as high as 2.0 wt. %, 3.0 wt. %, 4.0 wt. %, 5.0 wt. %, 6.0 wt. %, or within any range encompassing any two of these values as endpoints, based on to total weight of the catalyst. One suitable promoted chromium catalyst is a zinc/chromia catalyst which is based on chromia and includes an amount of zinc as a co-catalyst, for example, JM 62-3M catalyst available from Johnson Matthey. Prior to use, a fluorination treatment of the catalyst may be conducted using anhydrous HF under conditions effective to convert a portion of metal oxides into corresponding metal fluorides.

The above chromium-based catalysts may also be low chromium (VI) catalysts, having a total content of chromium (VI) oxide in an amount of about 5,000 ppm or less, about 2,000 ppm or less, about 1,000 ppm or less, about 500 ppm or less, about 250 ppm or less, or about 100 ppm or less based on total chromium oxides in the chromium oxide catalyst.

In addition to chromium-based catalysts, other suitable catalysts include alumina, iron oxide, magnesium oxide, zinc oxide, nickel oxide, cobalt oxide, aluminum fluoride or metal fluorides such as iron fluoride, magnesium fluoride, zinc fluoride, nickel fluoride, cobalt fluoride, fluorinated alumina, fluorinated iron oxide, fluorinated magnesium oxide, fluorinated nickel oxide, fluorinated cobalt oxide, titanium fluorides, molybdenum fluorides, aluminum oxyfluorides, and combinations of the foregoing. Prior to use, a fluorination treatment of catalyst containing metal oxide(s) is conducted using anhydrous HF under conditions effective to convert a portion of metal oxide(s) into corresponding metal fluoride(s). Pentavalent antimony, niobium, arsenic and tantalum halides are commercially available, and mixed halides thereof are created in situ upon reaction with HF. Antimony pentachloride is preferred because of its low cost and availability. Pentavalent antimony mixed halides of the formula SbClₙF₅₋ₙ where n is 0 to 5 are more preferred. The fluorination catalysts preferably have a purity of at least about 97%. Although the amount of fluorination catalyst used may vary widely, using from about 5 to about 50%, or preferably from about 10 to about 25% by weight catalyst, relative to the organics is suitable.

Following the dehydrofluorination reaction, crude product stream 18 is conveyed to separation device 20 for HF removal.

The composition in stream 18 comprises the crude products of the reactor which may include CFC-113, HFC-245fa, HFO-1234ze(Z), HFO-1234ze(E), CFC-114, light impurities, heavy impurities, and HF. HF may be present in stream 18 in an amount as little as 5 wt. %, 6 wt. %, 7 wt. %, 8 wt. %, 9 wt. %, 10 wt. %, 11 wt. %, 12 wt. %, 13 wt. %, 14 wt. %, 15 wt. %, or as great as 16 wt. %, 17 wt. %, 18 wt. %, 19 wt. %, 20 wt. %, 21 wt. %, 22 wt. %, 23 wt. %, 24 wt. %, 25 wt. %, or within any range encompassing any two of these values as endpoints, based on the total weight of the stream's composition.

Separation device 20 may be any separation device suitable for decreasing the level of HF in the composition of stream 18. In one embodiment, the HF may be recovered using water or caustic scrubbers or contacting with a metal salt such as potassium fluoride or sodium fluoride. In another embodiment, the HF may be recovered by passing the composition through a sulfuric acid extractor, desorbing the extracted HF from the sulfuric acid, and then distilling the desorbed hydrogen fluoride. The separation may be conducted by adding sulfuric acid to the mixture while the mixture is in either the liquid or gaseous states. In an alternate embodiment, the recovering of HF from the mixture may be conducted in a gaseous phase by a continuous process of introducing a stream of sulfuric acid to stream 18. This may be conducted in a standard scrubbing tower by flowing a stream of sulfuric acid countercurrent to stream 18. In another embodiment, HF may be removed by adsorption onto carbon molecular sieves, HF-polymer gel, membranes, or zeolites.

After removal of HF, stream 22 is condensed in condenser 24 to produce stream 26.

The composition in stream 22 may comprise the crude products of the reactor which include CFC-113, HFC-245fa, HFO-1234ze(Z), HFO-1234ze(E), CFC-114, light components, heavy components, and a reduced amount of HF. HF may be present in stream 22 in an amount of less than 5 wt. %, less than 4 wt. %, less than 3 wt. %, less than 2 wt. %, less than 1 wt. %, less than 0.5 wt. %, or less than 0.1 wt. %.

Condenser 24 may be operated at a pressure as low as 0 psia, 10 psia, 20 psia, 30 psia, 40 psia, 50 psia, 60 psia, 70 psia or as high as 80 psia, 90 psia, 100 psia, 110 psia, 120 psia, 130 psia, 140 psia, 150 psia, or within any range encompassing any two of these values as endpoints. Condenser 24 may be operated at a temperature as low as −20° C., −10° C., 0° C., 10° C., or as high as 20° C., 40° C., 50° C., 60° C., or within any range encompassing any two of these values as endpoints.

Stream 26 is fed into separation device 28 which separates the reaction mixture into stream 30 and waste stream 52 comprising light impurities.

The term “light impurities” as used herein refers to impurities or azeotropes that have a boiling point lower than −19° C., i.e., a lower boiling point than that of HFO-1234ze(E). Examples of light impurities include but are not limited to 3,3,3-trifluoropropyne (TFPy), TFPy/water azeotropes, TFPy/HF azeotropes, HFO-1234ze(E)/water azeotropes, HFO-1234ze(E)/HF azeotropes, HFO-1234yf (2,3,3,3-tetrafluoropropene), HFC-245cb (1,1,1,2,2-pentafluoropropane), HFC-152a (1,1-difluoroethane), HFC-134a (1,1,1,2-tetrafluoroethane), HFC-125 (pentafluoroethane), HFO-1225ye(Z) (1,2,3,3,3,-pentafluoropropene), HFO-1234zc (1,1,3,3,3-pentafluoropropene), and HFO-1234zf (2,3,3,3-tetrafluoropropene).

The composition in stream 26 may comprise CFC-113, HFC-245fa, HFO-1234ze(Z), HFO-1234ze(E), CFC-114, light impurities, heavy impurities, and a reduced amount of HF. The amount of light impurities in stream 26 may be as little as 0.01 wt. %, 0.02 wt. %, 0.03 wt. %, 0.04 wt. %, 0.05 wt. %, or as great as 0.06 wt. %, 0.07 wt. %, 0.08 wt. %, 0.09 wt. %, 0.1 wt. %, 0.5 wt. %, 1 wt. %, or within any range encompassing any two of these values as endpoints, based on the total weight of the stream's composition.

Separation device 28 may be any device suitable for separating light impurities from the reaction mixture. Examples of suitable devices include but are not limited to batch or continuous fractional distillation columns, spinning band distillation equipment, or wiped film evaporators. Waste stream 29 comprises light impurities which may be removed from the reaction mixture.

Stream 30 is fed into separation device 32 which affords crude product stream 44 comprising HFO-1234ze(E) and stream 34.

Stream 30 may comprise CFC-113, HFC-245fa, HFO-1234ze(Z), HFO-1234ze(E), CFC-114, a reduced amount of light impurities, heavy impurities, and a reduced amount of HF. The amount of light impurities in stream 30 may be less than 0.01 wt. %, 0.009 wt. %, 0.008 wt. %, 0.007 wt. %, 0.006 wt. %, 0.005 wt. %, 0.004 wt. %, 0.003 wt. %, 0.002 wt. %, or 0.001 wt. %.

Separation device 32 may be any separation device suitable for separating HFO-1234ze(E) from the mixture. Examples of suitable devices include but are not limited to batch or continuous fractional distillation columns, spinning band distillation equipment, or wiped film evaporators.

Stream 44, also referred to herein as the second product stream, is optionally fed into separation device 46 which reduces the level of CFC-114 to afford the purified final product HFO-1234ze(E) in stream 48. Stream 47 comprises CFC-114 which may be removed from the system for disposal.

Stream 44 may comprise CFC-114 and HFO-1234ze(E). The amount of CFC-114 may be as little as 1000 ppm or less, 900 ppm or less, 800 ppm or less, 700 ppm or less, 600 ppm or less, 500 ppm or less, 400 ppm or less, 300 ppm or less, 200 ppm or less, 100 ppm or less, or 50 ppm or less.

Stream 48 is a purified product stream which may comprise HFO-1234ze(E) and CFC-114 in an amount less than 0.001 wt. %, less than 0.0005 wt. %, or less than 0.00001 wt. %.

Separation device 46 may be any separation device suitable for decreasing the amount of CFC-114 in HFO-1234ze(E). In one embodiment, the separation devices may comprise a zeolite that exploits structural or size differences between the molecules. Suitable zeolites include sodium silicoaluminate, zeolite type X, AW-500, 3 A molecular sieves, 4 A molecular sieves, 5 A molecular sieves, activated carbon, or activated carbon molecular sieves. In one embodiment, the separation devices may comprise a zeolite that exploits the difference in dipole moment between the molecules. In one embodiment, the separation device may comprise an extractant to exploit the different solubility between the molecules. Suitable extractants include but are not limited to pentane, hexane, ethyl acetate, dichloromethane, chloroform, tetrahydrofuran, methanol, or water. In one embodiment, the separation device may comprise an azeotropic distillation apparatus. In one embodiment, the separation device may comprise an apparatus to pass the mixtures in vapor phase through mineral oil at a velocity low enough to effect dissolution of undesired CFC impurities in mineral oil and a knockout pot to eliminate oil from the desired component.

Stream 34, also referred to herein as the third product stream, is fed into distillation apparatus 36 which affords a bottoms stream comprising heavy impurities in stream 42 for removal, and an overhead recycle stream 50 which is fed back into the feed stream.

As used herein, the term “heavy impurities” refers to impurities or azeotropes that have a boiling point higher than −19° C., i.e., a higher boiling point than that of HFO-1234ze(E). Examples of heavy impurities include but are not limited to HCFO-1233ze(E) (trans-1-chloro-3,3,3-trifluoro-propene), HCFO_1233zd(Z) (cis-1-chloro-3,3,3-trifluoro-propene), HCFC-244fa (3-chloro-1,1,1,3-tetrafluoropropane), and CFC-113.

Stream 34 may comprise CFC-113, HFC-245fa, and HFO-1234ze(Z), and less than 0.001 wt. % HFO-1234ze(E).

Distillation apparatus 36 may be used to separate and purge heavy impurities from the system. The use of apparatus 36 may be done in a continuous or batch process. Apparatus 36 may be operated at a pressure as low as 0 psia, 10 psia, 20 psia, 30 psia, 40 psia, 50 psia, 60 psia, 70 psia or as high as 80 psia, 90 psia, 100 psia, 110 psia, 120 psia, 130 psia, 140 psia, 150 psia, or within any range encompassing any two of these values as endpoints. Apparatus 36 may be operated at a temperature as low as 10° C., 20° C., 30° C., 40° C., 50° C., or as high as 60° C., 70° C., 80° C., 90° C., 100° C., 110° C., or within any range encompassing any two of these values as endpoints.

Waste stream 42 comprises heavy impurities such as CFC-113 which may be removed from the system as waste, where the amount of CFC-113 in stream 42 may be 1 wt. % or higher, 2.5 wt. % or higher, 4 wt. % or higher, to 6 wt. % or lower, 7.5 wt. % or lower, or 10 wt. % or lower or within any range encompassing any two of these values as endpoints.

Recycle stream 50 may comprise CFC-113, HFC-245fa, an azeotrope of CFC-113 and HFC245fa, HFO-1234ze(Z), and HFO-1234ze(E).

The heating system for the reactor and distillation may comprise any suitable heating medium capable of achieving and/or maintaining the temperatures required by the process. Suitable heating media may include molten salt, hot oil, steam, and electric heaters (resistance or induction), among others, for example.

### IV. Purging CFC-113 from the Recycle Stream

It has been found, as shown in Example 2A below, that it is not necessary to focus solely on removing CFC-114 from the HFO-1234ze(E) product or CFC-113 from the HFC-245fa feed. Instead, CFC-113 can be purged from an intermediate stream in the process where the separation is easier so that smaller, less expensive equipment can be used with lower energy consumption and lower yield losses. Subjecting this intermediate stream to a separation and distillation provides a recycle stream comprising unconverted HFC-245fa, HFO-1234ze(Z), HFO-1234ze(E), and reduced amounts of CFC-113 which can be fed back into the reactor, and a waste stream comprising impurities which can be removed from the system.

As discussed further in section VI below, it has been found that CFC-113 and HFC-245fa form a homogenous minimum boiling point azeotropic mixture at about 3.5 wt. % CFC-113 and about 96.5 wt. % HFC-245fa and at a temperature of about 14.44° C.±0.3° C. and a pressure of about 14.29 psia±0.3 psia.

This process follows the same flow as outlined in Section III and in FIG. 1, but the level of CFC-113 is allowed to build up in the recycle stream before being purged from the system. Because of the discovered azeotropic behavior of the CFC-113 and HFC-245fa mixture, it may be beneficial to remove CFC-113 from the recycle stream in which the CFC-113 has built up to 1 wt. % or higher, 1.5 wt. % or higher, 2 wt. % or higher, 2.5 wt. % or higher, 3 wt. % or higher, 3.5 wt. % or higher, 4 wt. % or higher, or 5 wt. % or higher. This method strikes a balance between decreasing CFC impurities to acceptable levels while minimizing unnecessary separations and reducing yield loss. Allowing the concentration of CFC-113 to build up before it is removed avoids trying to separate the components at the pinch point in the CFC-113 and HFC-245fa mixture.

### V. Conducting the Separation at Higher Pressures

It has also been found, as shown in Examples 2B and 5 below, that the vapor liquid equilibrium concerning the formation of an azeotrope or azeotrope like mixture of CFC-113 and HFC-245fa is pressure dependent, and that the azeotropic behavior between CFC-113 and HFC-245fa can be substantially or entirely avoided when a mixture including CFC-113 and HFC-245fa is exposed to a pressure greater than about 17 psia, for example, by operating separation stage of the present process at such an elevated pressure to avoid formation of the azeotrope.

Operating the separation step of CFC-113 from HFC-245fa at these higher pressures advantageously avoids any need to allow CFC-113 to concentrate in the recycle stream before removing it per the method discussed in section IV above. In addition, operating at higher pressures makes the final separation of CFC-113 from HFC-245fa easier because it avoids the formation of the CFC-113/HFC-245fa azeotrope such that these components may be separated effectively according to the difference in their boiling points.

This process follows the same flow outlined in Section III and in FIG. 1, but with higher pressures in the distillation apparatus, increased amounts of CFC-113 in the waste stream, and decreased amounts of CFC-113 in the recycle stream. All other parameters including the reactor set up and temperature conditions are the same as what is described above in section III.

The distillation apparatus 36 may be operated at a pressure of 17 psia or higher, 18 psia or higher, 19 psia or higher, 20 psia or higher, 25 psia or higher, 30 psia or higher, 35 psia or higher, 40 psia or higher, 45 psia or higher, 50 psia or higher, 55 psia or higher, 60 psia or higher, 65 psia or higher, 70 psia or higher, 75 psia or higher, 80 psia or higher, 85 psia or higher, 90 psia or higher, 95 psia or higher, 100 psia or higher, 105 psia or higher, 110 psia or higher, 115 psia or higher, 120 psia or higher, 125 psia or higher, 130 psia or higher, 135 psia or higher, 140 psia or higher, 145 psia or higher, or 150 psia or higher, or within any range encompassing any two of these values as endpoints.

Waste stream 42 comprises heavy impurities such as CFC-113 which may be removed from the system as waste, where the amount of CFC-113 in stream 42 may be from 1 wt. % or higher, 2 wt. % or higher, 3 wt. % or higher, 4 wt. % or higher, 5 wt. % or higher, 6 wt. % or higher, 7 wt. % or higher, 8 wt. % or higher, 9 wt. % or higher, 10 wt. % or higher, 15 wt. % or higher, 20 wt. % or higher, 25 wt. % or higher, 30 wt. % or higher, 35 wt. % or higher, 40 wt. % or higher, 45 wt. % or higher, 50 wt. % or higher, 55 wt. % or higher, 60 wt. % or higher, 65 wt. % or higher, 70 wt. % or higher, 75 wt. % or higher, 80 wt. % or higher, 85 wt. % or higher, 90 wt. % or higher, 95 wt. % or higher, 100 wt. %, or within any range encompassing any two of these values as endpoints.

Recycle stream 50 may comprise CFC-113, HFC-245fa, an azeotrope of CFC-113 and HFC245fa, HFO-1234ze(Z), and HFO-1234ze(E). The amount of CFC-113 in the recycle stream may be 10 wt. % or lower, 9 wt. % or lower, 8 wt. % or lower, 7 wt. % or lower, 6 wt. % or lower, 5 wt. % or lower, 4 wt. % or lower, 3 wt. % or lower, 2 wt. % or lower, 1 wt. % or lower, 0.5 wt. % or lower, 0.1 wt. % or lower, or within any range encompassing any two of these values as endpoints.

### VI. Azeotropic Composition Comprising HFC-245fa and CFC-113

The present inventors have found experimentally that HFC-245fa and CFC-113 form an azeotrope or azeotrope-like composition at pressures below 25 psia. The discovery of this azeotropic mixture is pertinent to the process for converting HFC-245fa to HFO-1234ze(E). CFC-113 is an undesirable impurity that may be found in the HFC-245fa feedstock and that may convert to CFC-114 during the dehydrofluorination reaction to produce HFO-1234ze(E). Therefore, methods to separate mixtures of HFC-245fa and CFC-113 are needed to prevent the buildup of CFCs throughout the HFO-1234ze(E) process.

An “azeotrope” composition is a unique combination of two or more components. An azeotrope composition can be characterized in various ways. For example, at a given pressure, an azeotrope composition boils at a constant characteristic temperature which is either greater than the higher boiling point component (maximum boiling azeotrope) or less than the lower boiling point component (minimum boiling azeotrope). At this characteristic temperature the same composition will exist in both the vapor and liquid phases. The azeotrope composition does not fractionate upon boiling or evaporation. Therefore, the components of the azeotrope composition cannot be separated during a phase change.

An azeotrope composition is also characterized in that at the characteristic azeotrope temperature, the bubble point pressure of the liquid phase is identical to the dew point pressure of the vapor phase.

The behavior of an azeotrope composition is in contrast with that of a non-azeotrope composition in which during boiling or evaporation, the liquid composition changes to a substantial degree.

For the purposes of the present disclosure, an azeotrope composition is characterized as that composition which boils at a constant characteristic temperature, the temperature being lower (a minimum boiling azeotrope) than the boiling points of the two or more components, and thereby having the same composition in both the vapor and liquid phases.

One of ordinary skill in the art would understand however that at different pressures, both the composition and the boiling point of the azeotrope composition will vary to some extent. Therefore, depending on the temperature and/or pressure, an azeotrope composition can have a variable composition. The skilled person would therefore understand that composition ranges, rather than fixed compositions, can be used to define azeotrope compositions. In addition, an azeotrope may be defined in terms of exact weight percentages of each component of the compositions characterized by a fixed boiling point at a specified pressure.

An “azeotrope-like” composition is a composition of two or more components which behaves substantially as an azeotrope composition. Thus, for the purposes of this disclosure, an azeotrope-like composition is a combination of two or more different components which, when in liquid form under given pressure, will boil at a substantially constant temperature, and which will provide a vapor composition substantially identical to the liquid composition undergoing boiling.

For the purposes of this disclosure, the azeotrope or azeotrope-like composition comprising HFC-245fa and CFC-113 is a composition or range of compositions which boils at a temperature range of about 14.44° C.±0.3° C. at a pressure of about 14.29 psia±0.3 psia.

Azeotrope or azeotrope-like compositions can be identified using a number of different methods. For the purposes of this disclosure the azeotrope or azeotrope-like composition is identified experimentally using an ebulliometer (Walas, Phase Equilibria in Chemical Engineering, Butterworth-Heinemann, 1985, 533-544). An ebulliometer is designed to provide extremely accurate measurements of the boiling points of liquids by measuring the temperature of the vapor-liquid equilibrium.

The boiling points of each of the components alone are measured at a constant pressure. As the skilled person will appreciate, for a binary azeotrope or azeotrope-like composition, the boiling point of one of the components of the composition is initially measured. The second component of the composition is then added in varying amounts and the boiling point of each of the obtained compositions is measured using the ebulliometer at said constant pressure.

The measured boiling points are plotted against the composition of the tested composition, for example, for a binary azeotrope, the amount of the second component added to the composition, (expressed as either weight % or mole %). The presence of an azeotrope composition can be identified by the observation of a maximum or minimum boiling temperature which is greater or less than the boiling points of any of the components alone.

As the skilled person will appreciate, the identification of the azeotrope or azeotrope-like composition is made by the comparison of the change in the boiling point of the composition on addition of the second component to the first component, relative to the boiling point of the first component. Thus, it is not necessary that the system be calibrated to the reported boiling point of the particular components in order to measure the change in boiling point.

As previously discussed, at the maximum or minimum boiling point, the composition of the vapor phase will be identical to the composition of the liquid phase. The azeotrope-like composition is therefore that composition of components which provides a substantially constant minimum or maximum boiling point, that is a boiling point of about 14.44° C.±0.3° C. at a pressure of about 14.29 psia±0.3 psia, at which substantially constant boiling point the composition of the vapor phase will be substantially identical to the composition of the liquid phase.

The azeotrope or azeotrope-like composition, at a boiling point of about 14.44° C.±0.3° C. at a pressure of about 14.29 psia±0.3 psia, comprises, consists essentially of, or consists of, from about 1 wt. % to about 15 wt. wt. % CFC-113, from about 1 wt. % to about 10 wt. % CFC-113, from about 1 wt. % to about 5 wt. % CFC-113, or about 3.5 wt. % CFC-113, and from about 85 wt. % to about 99 wt. % HFC-245fa, from about 90 wt. % to about 99 wt. % HFC-245fa, from about 95 wt. % to about 99 wt. % HFC-245fa, or about 96.5 wt. % HFC-245fa.

In other words, the azeotrope or azeotrope-like composition, at a boiling point of about 14.44° C.±0.3° C. at a pressure of about 14.29 psia±0.3 psia, comprises, consists essentially of, or consists of from about 1 wt. % to about 15 wt. % CFC-113 and from about 85 wt. % to about 99 wt. % HFC-245fa, or from about 1 wt. % to about 10 wt. % CFC-113 and from about 90 wt. % to about 99 wt. % HFC-245fa, or from about 1 wt. % to about 5 wt. % CFC-113 and about 95 wt. % to about 99 wt. % HFC-245fa, or about 3.5 wt. % CFC-113 and about 96.5 wt. % HFC-245fa.

Stated alternatively, the azeotrope or azeotrope-like composition, at a boiling point of about 14.44° C.±0.3° C. at a pressure of about 14.29 psia±0.3 psia, comprises, consists essentially of, or consists of, as little as about 1 wt. % CFC-113 or as great as about 5 wt. %, about 10 wt. %, or about 15 wt. % CFC-113, or within any range defined between any two of the foregoing values, and the azeotrope or azeotrope-like composition comprises, consists essentially or, or consists of, as little as about 85 wt. %, about 90 wt. %, about 95 wt. % HFC-245fa or as great as about 99 wt. % HFC-245fa, or within any range defined between any two of the foregoing values.

The azeotrope or azeotrope-like composition of the present disclosure has a boiling point of about 14.44° C.±0.3° C. at a pressure of about 14.29 psia±0.3 psia.

It has also been found that the azeotrope or azeotrope-like composition is pressure dependent. The constant minimum or maximum boiling point behavior is observed at pressures below about 17 psia and, at pressures above about 17 psia, the azeotrope or azeotrope-like behavior is not observed.

For example, the azeotrope or azeotrope-like composition may be present at pressures below about 17 psia such as 16 psia or lower, 15 psia or lower, 14 psia or lower, 13 psia or lower, 12 psia or lower, 11 psia or lower, 10 psia or lower, 9 psia or lower, 8 psia or lower, 7 psia or lower, 6 psia or lower, 5 psia or lower, 4 psia or lower, 3 psia or lower, 2 psia or lower, 1 psia or lower, or within any range encompassed by any two of the foregoing values as endpoints.

The azeotrope or azeotrope-like composition may not be present at pressures above about 17 psia such as 18 psia or higher, 19 psia or higher, 20 psia or higher, 21 psia or higher, 22 psia or higher, 23 psia or higher, 24 psia or higher, 25 psia or higher, 26 psia or higher, 27 psia or higher, 28 psia or higher, 29 psia or higher, 30 psia or higher, 31 psia or higher, 32 psia or higher, 33 psia or higher, 34 psia or higher, 35 psia or higher, 36 psia or higher, or within any range encompassed by any two of the foregoing values as endpoints.

As used herein, the phrase “within any range encompassed by any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

### EXAMPLES

### Example 1—Ebulliometer Study

An ebulliometer was used to measure azeotrope and azeotrope-like compositions of HFC-245fa and CFC-113. The ebulliometer included a vacuum jacketed glass vessel which was sealed at the bottom and open to the atmosphere at the top. The top, or condenser jacket, of the ebulliometer was filled with a mixture of dry ice and ethanol to attain a temperature of about −72° C., which is significantly lower than the normal boiling points of 15.3° C. for 1,1,1,3,3-pentafluoropropane (HFC-245fa) and 47.5° C. for 1,1,2-trichloro-trifluoroethane (CFC-113) at a pressure of 14.40 psia. In this manner, it was ensured that all vapors in the system were condensed and flowed back into the ebulliometer such that the liquid and vapor phases were in equilibrium. A quartz-platinum thermometer with an accuracy of ±0.002° C. was inserted inside the glass vessel and used to determine the temperature of the condensed vapor corresponding to the equilibrium boiling point of the mixture. Boiling chips were used to assist with maintaining a smooth boiling of the mixture in the ebulliometer.

The following procedure was used:
1. The quartz thermometer was immersed into a long dewar which contained an ice/water slurry, and it was verified that the thermometer read 0° C. The dewar was deep enough so that at least ¾ the length of the thermometer shaft was immersed in the ice/water. The thermometer resistance was recorded in ohms.
2. The condenser jacket was loaded to ¼ full with ethanol. The condenser jacket was cooled by slowly introducing dry ice to avoid boiling over and/or splashing of the ethanol.
3. A known amount of CFC-113 or HFC-245fa was added to the ebulliometer and brought to a vigorously refluxing condition. The temperature and atmospheric pressure were recorded using a barometer with a temperature indicator.

The measurement was carried out in two steps. In a first step, about 24.50 g of CFC-113 having a purity of 99.88 area % as determined by gas chromatography (GC) was first introduced to the ebulliometer by weighing the container before and after the addition using a balance having an accuracy of ±0.01 g. The liquid was brought to a boil and the equilibrium temperature of the CFC-113 was recorded at the recorded barometric pressure. Then, HFC-245fa having a purity of 99.99 area % as determined by gas chromatography (GC) was introduced in small increments into the ebulliometer and the equilibrium temperature of the condensed liquid mixture was recorded.

In a second step, about 16.09 g of HFC-245fa having a purity of 99 area % as determined by gas chromatography (GC) was introduced to the ebulliometer by weighing the container before and after the addition using a balance having an accuracy of ±0.01 g. The liquid was brought to a boil and the equilibrium temperature of the HFC-245fa was recorded at the recorded barometric pressure. Then, CFC-113 having a purity of 99.88 area % as determined by gas chromatography (GC) was introduced in small increments into the ebulliometer and the equilibrium temperature of the condensed liquid mixture was recorded.

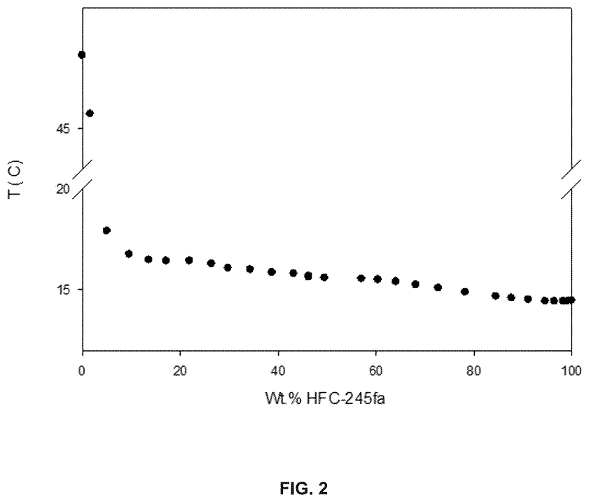

Data from the above first and second steps was combined to complete the composition range data from 0 to 100 weight percent of each of the HFC-245fa and the CFC-113 presented below in Table 2, which shows a minimum in temperature which indicates that an azeotrope had been formed, and this data is also presented in graphic form in FIG. 2. The data suggests the formation of a weak minimum boiling azeotrope at about 3.5 wt. % CFC-113 at a pressure of 14.29 Psia. The azeotropic temperature is 14.44° C. which is below the boiling point of HFC-245fa.

TABLE 2
Ebulliometer Study of HFC-245fa/CFC-113 at P = 14.29 Psia

| T (° C.) | Wt. % HFC-245fa | Wt. % CFC-113 |
|---|---|---|
| 14.47 | 100.00 | 0.00 |
| 14.46 | 99.26 | 0.74 |
| 14.45 | 98.26 | 1.74 |
| 14.44 | 96.45 | 3.55 |
| 14.45 | 94.59 | 5.41 |
| 14.52 | 91.13 | 8.87 |
| 14.60 | 87.70 | 12.30 |
| 14.69 | 84.52 | 15.48 |
| 14.90 | 78.22 | 21.78 |
| 15.10 | 72.76 | 27.24 |
| 15.26 | 68.17 | 31.83 |
| 15.41 | 64.09 | 35.91 |
| 15.51 | 60.43 | 39.57 |
| 15.55 | 57.07 | 42.93 |
| 15.71 | 53.48 | 46.52 |
| 15.61 | 49.49 | 50.51 |
| 15.64 | 46.27 | 53.73 |
| 15.81 | 43.20 | 56.80 |
| 15.86 | 38.77 | 61.23 |
| 16.01 | 34.30 | 65.70 |
| 16.08 | 29.80 | 70.20 |
| 16.29 | 26.40 | 73.60 |
| 16.44 | 21.92 | 78.08 |
| 16.43 | 17.15 | 82.85 |
| 16.49 | 13.63 | 86.37 |
| 16.76 | 9.61 | 90.39 |
| 17.92 | 5.07 | 94.93 |
| 45.37 | 1.63 | 98.37 |
| 46.82 | 0.00 | 100.00 |

### Example 2A—Process for Removing CFC-114 in HFO-1234ze(E) By Purin CFC-113 from the Recycle Stream

Example 2A provides the material balance for each stream in the process depicted in FIG. 1.

TABLE 3
Material balance for FIG. 1.

| | Streams (mass per time) | | | | | | | |
|---|---|---|---|---|---|---|---|---|
| Components | 14 | 18 | 22 | 30 | 44 | 38 | 42 | 50 |
| CFC-113 | 0.070 | 1.065 | 1.065 | 1.065 | — | 1.065 | 0.065 | 1.000 |
| HFC-245fa | 99.930 | 80.000 | 80.000 | 80.000 | — | 80.000 | 1.000 | 79.000 |
| HFO- | — | 19.000 | 19.000 | 19.000 | — | 19.000 | 0.000 | 19.000 |
| 1234ze(Z) | | | | | | | | |
| CFC-114 | — | 0.005 | 0.005 | 0.005 | 0.005 | — | — | — |
| HFO- | — | 84.975 | 84.975 | 84.975 | 83.975 | 1.000 | 0.000 | 1.000 |
| 1234ze(E) | | | | | | | | |
| Lites | — | 0.040 | 0.040 | — | — | — | — | — |
| Components | | | | | | | | |
| HF | — | 14.915 | — | — | — | — | — | — |

As shown in FIG. 1, the CFC-113 impurity is removed from the recycle stream of the HFC-245fa to HFO-1234ze(E) process via distillation at a pressure below 17 psia. The recycle stream 50 mainly comprises unconverted HFC-245fa, HFO-1234ze(Z), 1234ze(E), and CFC-113. Due to the difficulty involved in separating CFC-113 from HFC-245fa, it is beneficial to remove 113 from a recycle stream in which the component has built up to 1 wt. % or higher. Because CFC-113 is a high boiling impurity, it can be removed in the heavy impurities purge stream 42.

### Example 2B—Process for Removing CFC-114 in HFO-1234ze(E) by Operating at Higher Pressures

Example 2B provides the material balance for each stream in the process depicted in FIG. 1. Example 2B is different from 2 A in that the removal of CFC-113 via distillation (block 36) is performed at a pressure above 17 psia, which doesn't require 113 to build up in the recycle stream 50.

TABLE 4
Material balance for FIG. 1.

| | Streams (mass per time) | | | | | | | |
|---|---|---|---|---|---|---|---|---|
| Components | 14 | 18 | 22 | 30 | 44 | 38 | 42 | 50 |
| CFC-113 | 0.070 | 0.169 | 0.169 | 0.169 | — | 0.169 | 0.069 | 0.100 |
| HFC-245fa | 99.930 | 80.000 | 80.000 | 80.000 | — | 80.000 | 1.000 | 79.000 |
| HFO- | — | 19.000 | 19.000 | 19.000 | — | 19.000 | 0.000 | 19.000 |
| 1234ze(Z) | | | | | | | | |
| CFC-114 | — | 0.001 | 0.001 | 0.001 | 0.001 | — | — | — |
| HFO- | — | 85.875 | 85.875 | 85.875 | 83.975 | 1.900 | 0.000 | 1.900 |
| 1234ze(E) | | | | | | | | |
| Lites | — | 0.040 | 0.040 | — | — | — | — | — |
| Components | | | | | | | | |
| HF | — | 14.915 | — | — | — | — | — | — |

As shown in FIG. 1, the CFC-113 impurity is removed from the recycle stream of the HFC-245fa to HFO-1234ze(E) process via distillation at a pressure above 17 psia. The recycle stream 50 mainly comprises unconverted HFC-245fa, HFO-1234ze(Z), 1234ze(E), and CFC-113. Because the azeotrope between 113 and 245fa does not exist at this pressure, is it not required to have the 113 up to 1 wt. % or higher. This reduces the amount of 113 being fed to and the amount of 114 produced by the reactor (block 16). Because CFC-113 is a high boiling impurity, it can be removed in the heavy impurities purge stream 42.

### Example 3—Separating Mixtures of CFC-113/HFC-245fa

### Example 3A—Adsorbents

Referring back to FIG. 1, CFC-113 is removed from a stream comprising CFC-113 and HFC-245fa by passing through an adsorbent device that comprises molecular sieves. The amount of CFC-113 is thereby reduced to an acceptable level for the HFC-245fa feed stream.

### Example 3B—Liquid Extraction

Referring back to FIG. 1, CFC-113 is removed from a stream comprising CFC-113 and HFC-245fa by liquid extraction using a mixture of hexanes and ethyl acetate. The amount of CFC-113 is thereby reduced to an acceptable level for the HFC-245fa feed stream.

### Example 3C—Mineral Oil Extraction

Referring back to FIG. 1, CFC-113 is removed from a stream comprising CFC-113 and HFC-245fa by mineral oil extraction. A stream of HFC-245fa comprising 0.50 wt. % CFC-113 is vaporized and fed to the bottom of a packed column at a feed rate of about 2.2 lbs per hour for about 4 hours. A stream of mineral oil is fed continuously to the top of the same packed column at a feed rate of about 3.5 lbs per hour during the same time frame. A gaseous stream exiting the top of the column comprises HFC-245fa with less than 0.10 wt. % CFC-113. The concentration of CFC-113 in the mineral oil increases from non-detectable to about 0.25 wt. %.

### Example 4—Separating Mixtures of CFC-114/HFO-1234ze(E)

### Example 4A—Adsorbents

Referring back to FIG. 1, CFC-114 is removed from a stream comprising CFC-114 and HFO-1234ze(E) by passing through an adsorbent device that comprises molecular sieves. The amount of CFC-114 is thereby reduced to an acceptable level for the HFO-1234ze(E) product stream.

### Example 4B—Liquid Extraction

Referring back to FIG. 1, CFC-114 is removed from a stream comprising CFC-114 and HFO-1234ze(E) by liquid extraction using a mixture of hexanes and ethyl acetate. The amount of CFC-114 is thereby reduced to an acceptable level for the HFO-1234ze(E) product stream.

### Example 4C—Mineral Oil Extraction

Referring back to FIG. 1, CFC-114 is removed from a stream comprising CFC-114 and HFO-1234ze(E) by mineral oil extraction. A stream of HFO-1234ze(E) containing 0.20 wt. % CFC-114 impurity is vaporized and fed to the bottom of a packed column at a feed rate of about 2.1 lbs per hour for about 4 hours. A stream of mineral oil is fed continuously to the top of the same packed column at a feed rate of about 3.3 lbs per hour during the same time frame. A gaseous stream exiting the top of the column comprises HFO-1234ze(E) with less than 0.050 wt. % CFC-114. The concentration of CFC-114 in the mineral oil increases from non-detectable to about 0.095 wt. %.

### Example 5—Ebuilliometer Study of HFC-245fa and CFC-113 Mixtures at Different Pressures

HFC-245fa and CFC-113 were tested in an isobaric ebulliometer. A pressure controller was used to set the pressure and a RTD to measure the temperature. Charging and additions were done through a pump volumetrically. The following data sets are the results from these experiments. The conclusion was that the azeotrope composition shifts based on pressure and is predicted to break above 20.4 psia.

TABLE 5
HFC-245fa and CFC-113 Composition and Measured Temperatures

| 5 psia | | | 10 psia | | | 14.7 psia | | |
|---|---|---|---|---|---|---|---|---|
| R245fa | R113 | Temp | R245fa | R113 | Temp | R245fa | R113 | Temp |
| (mass %) | (mass %) | (C.) | (mass %) | (mass %) | (C.) | (mass %) | (mass %) | (C.) |
| 100.00% | 0.00% | −8.850 | 100.00% | 0.00% | 6.017 | 100.00% | 0.00% | 15.204 |
| 98.88% | 1.12% | −8.879 | 99.01% | 0.99% | 6.004 | 99.01% | 0.99% | 15.198 |
| 97.76% | 2.24% | −8.904 | 98.00% | 2.00% | 5.990 | 98.00% | 2.00% | 15.191 |
| 96.65% | 3.35% | −8.926 | 97.01% | 2.99% | 5.981 | 97.01% | 2.99% | 15.188 |
| 95.53% | 4.47% | −8.944 | 96.00% | 4.00% | 5.978 | 95.91% | 4.09% | 15.191 |
| 94.42% | 5.58% | −8.953 | 95.01% | 4.99% | 5.975 | 94.90% | 5.10% | 15.196 |
| 93.31% | 6.69% | −8.957 | 94.00% | 6.00% | 5.978 | 93.92% | 6.08% | 15.205 |
| 92.20% | 7.80% | −8.960 | 93.00% | 7.00% | 5.985 | 92.90% | 7.10% | 15.218 |
| 91.10% | 8.90% | −8.955 | 92.00% | 8.00% | 5.994 | 91.91% | 8.09% | 15.234 |
| 90.00% | 10.00% | −8.947 | 91.00% | 9.00% | 6.006 | 90.92% | 9.08% | 15.258 |
| 88.89% | 11.11% | −8.935 | 90.00% | 10.00% | 6.024 | 89.92% | 10.08% | 15.275 |
| 83.45% | 16.55% | −8.837 | 85.00% | 15.00% | 6.151 | 84.93% | 15.07% | 15.430 |
| 78.07% | 21.93% | −8.679 | 80.00% | 20.00% | 6.328 | 79.94% | 20.06% | 15.644 |

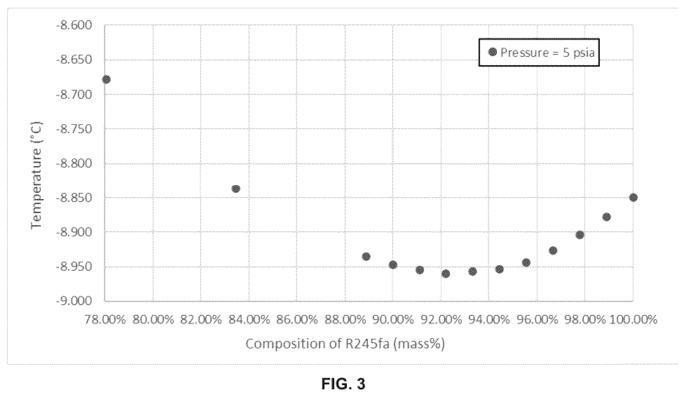

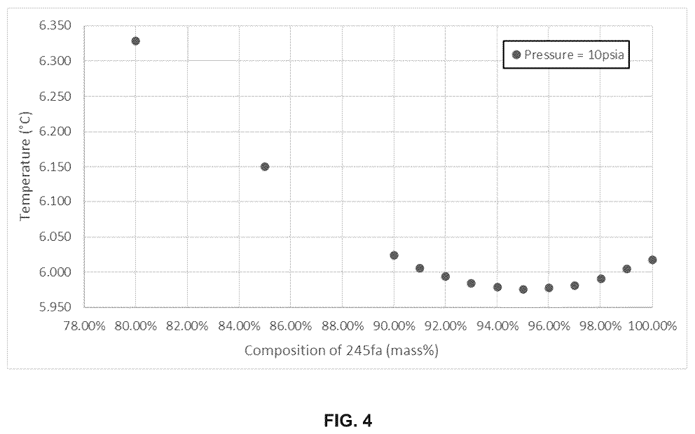

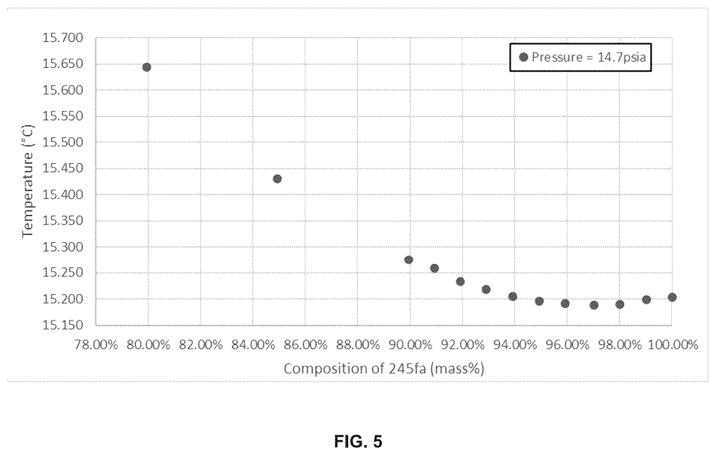

These data are plotted in FIGS. 3-5. FIG. 3 shows the minimum boiling azeotrope at 92.20 wt. % HFC-245fa at a pressure of 5 psia. FIG. 4 shows the minimum boiling azeotrope at 95.01 wt. % HFC-245fa at a pressure of 10 psia. FIG. 5 shows the minimum boiling azeotrope at 97.01 wt. % HFC-245fa at a pressure of 14.7 psia.

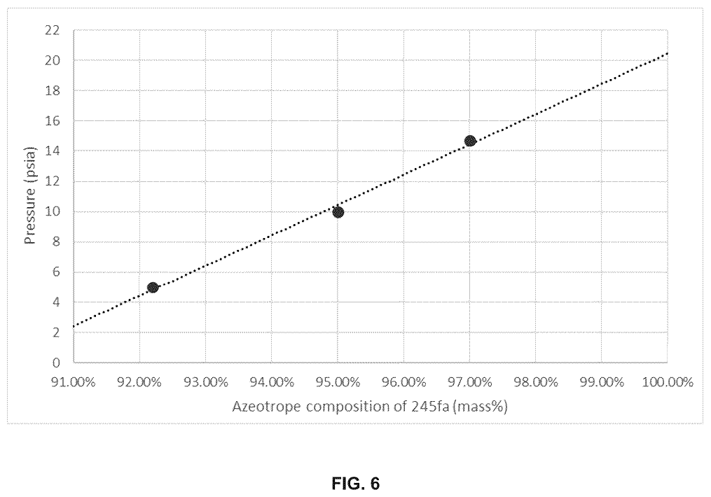

As the pressure increases through FIGS. 3 to 5, the azeotropic behavior becomes weaker, indicating that the azeotropic behavior between HFC-245fa and CFC-113 is pressure dependent. FIG. 6 plots pressure vs. the azeotrope composition of HFC-245fa (mass %) and shows that the locus of the azeotrope shifts with pressure.

### ASPECTS

Aspect 1 is a process for producing HFO-1234ze(E), comprising: reacting a feed stream comprising HFC-245fa and CFC-113 in a reactor in the presence of a catalyst to form a first product stream comprising HFO-1234ze(E), unreacted HFC-245fa, and CFC-113; separating the first product stream into a second product stream comprising HFO-1234ze(E) and a third product stream comprising unreacted HFC-245fa and CFC-113; distilling the third product stream to produce an overhead recycle stream and a bottoms stream, the recycle stream comprising unreacted HFC-245fa and a first amount of CFC-113, and the bottoms stream comprising a second amount CFC-113 greater than the first amount; and conveying the recycle stream back to the feed stream.
Aspect 2 is the process of Aspect 1, wherein the distillation is conducted at a pressure above 17 psia.
Aspect 3 is the process of Aspect 1 or 2, wherein the recycle stream includes less than 3 wt. % CFC-113.
Aspect 4 is the process of any of Aspects 1 to 3, wherein the distillation step comprises distilling the third product stream when the amount of CFC-113 is 1 wt. % or greater based on the total weight of the third product stream.
Aspect 5 is the process of any of Aspects 1 to 4, wherein an amount of CFC-113 in the recycle stream is 5 wt. % or less based on the total weight of the recycle stream.
Aspect 6 is the process of any of Aspects 1 to 5, wherein the first product stream additionally comprises hydrogen fluoride (HF) and is conveyed to a separation device to remove HF before entering the distillation apparatus to remove CFC-113.
Aspect 7 is the process of Aspect 6, wherein the separation device comprises at least one of a water or caustic scrubber, metal salts, a sulfuric acid extractor, a gaseous phase sulfuric acid extractor, a distillation tower, and combinations of the foregoing.
Aspect 8 is the process of any of Aspects 1 to 7, wherein the first product stream additionally comprises light impurities comprising at least one of 3,3,3-trifluoropropyne (TFPy), TFPy/water azeotropes, TFPy/HF azeotropes, HFO-1234ze(E)/water azeotropes, HFO-1234ze(E)/HF azeotropes, HFO-1234yf (2,3,3,3-tetrafluoropropene), HFC-245cb (1,1,1,2,2-pentafluoropropane), HFC-152a (1,1-difluoroethane), HFC-134a (1,1,1,2-tetrafluoroethane), HFC-125 (pentafluoroethane), HFO-1225ye(Z) (1,2,3,3,3,-pentafluoropropene), HFO-1234zc (1,1,3,3,3-pentafluoropropene), or HFO-1234zf (2,3,3,3-tetrafluoropropene) and combinations of the foregoing, and is conveyed to a separation device to remove the light impurities before entering the distillation apparatus to remove CFC-113.
Aspect 9 is the process of Aspect 8, wherein the separation device comprises at least one of a batch or continuous fractional distillation column, spinning band distillation equipment, a wiped film evaporator, and combinations of the foregoing.
Aspect 10 is the process of Aspect 8, wherein the level of light impurities is reduced to 1 wt. % or less based on the total weight of the intermediate stream.
Aspect 11 is the process of any one of Aspects 1 to 10, wherein the reactor is operated at at least one of the following conditions:
   (i) a temperature of about 205° C. to 370° C.;
   (ii) a pressure of about −15 to 100 psia; and
   (iii) a residence time of about 1 second to 100 seconds.
Aspect 12 is the process of Aspect 1, wherein the distillation apparatus is operated at at least one of the following conditions:
   (i) a temperature of about 10° C. to 105° C. and
   (ii) a pressure of about −15 to 150 psia
Aspect 13 is a process for removing CFC-113 from the HFC-245fa feed stream in the production of HFO-1234ze(E), comprising: feeding HFC-245fa to a separation device, the HFC-245fa further comprising a first amount of CFC-113; recovering, from the separation device, a purified HFC-245fa stream having a second amount of CFC-113 less than the first amount.
Aspect 14 is the process of Aspect 13, wherein the separation device comprises at least one of zeolites, liquid extractants, azeotropic distillation apparatuses, mineral oil, and combinations of the foregoing.
Aspect 15 is the process of Aspect 13, wherein the purified HFC-245fa stream comprises less than 0.05 wt. % CFC-113, based on a total weight of the purified HFC-245fa stream.
Aspect 16 is a process for removing CFC-114 from the HFO-1234ze(E) product stream in the HFO-1234ze(E) process comprising: feeding HFO-1234ze(E) to a separation device, the HFO-1234ze(E) further comprising a first amount of CFC-114; recovering, from the separation device, a purified HFO-1234ze(E) stream having a second amount of CFC-114 less than the first amount.
Aspect 17 is the process of Aspect 16, wherein the separation device comprises at least one of zeolites, liquid extractants, azeotropic distillation apparatuses, mineral oil, and combinations of the foregoing.
Aspect 18 is the process of Aspect 16, wherein the purified HFO-1234ze(E) stream comprises less than 0.01 wt. % CFC-114.
Aspect 19 is a composition comprising an azeotrope or azeotrope-like composition consisting essentially of effective amounts of CFC-113 and HFC-245fa.
Aspect 20 is the composition of Aspect 19, wherein the azeotrope or azeotrope-like composition consists essentially of about 3.5 wt. % CFC-113 and about 96.5 wt. % HFC-245fa.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

1. A process for producing HFO-1234ze(E), comprising:
reacting a feed stream comprising HFC-245fa and CFC-113 in a reactor in the presence of a catalyst to form a first product stream comprising HFO-1234ze(E), unreacted HFC-245fa, and CFC-113;
separating the first product stream into a second product stream comprising HFO-1234ze(E) and a third product stream comprising unreacted HFC-245fa and CFC-113;
distilling the third product stream to produce an overhead recycle stream and a bottoms stream, the recycle stream comprising unreacted HFC-245fa and a first amount of CFC-113, and the bottoms stream comprising a second amount CFC-113 greater than the first amount; and
conveying the recycle stream back to the feed stream.

2. The process of claim 1, wherein the distillation is conducted at a pressure above 17 psia.

3. The process of claim 1, wherein the recycle stream includes less than 3 wt. % CFC-113.

4. The process of claim 1, wherein the distillation step comprises distilling the third product stream when the amount of CFC-113 is 1 wt. % or greater based on the total weight of the third product stream.

5. The process of claim 1, wherein an amount of CFC-113 in the recycle stream is 5 wt. % or less based on the total weight of the recycle stream.

6. The process of claim 1, wherein the first product stream additionally comprises hydrogen fluoride (HF) and is conveyed to a separation device to remove HF before entering the distillation apparatus to remove CFC-113.

7. The process of claim 6, wherein the separation device comprises at least one of a water or caustic scrubber, metal salts, a sulfuric acid extractor, a gaseous phase sulfuric acid extractor, a distillation tower, and combinations of the foregoing.

8. The process of claim 1, wherein the first product stream additionally comprises light impurities comprising at least one of 3,3,3-trifluoropropyne (TFPy), TFPy/water azeotropes, TFPy/HF azeotropes, HFO-1234ze(E)/water azeotropes, HFO-1234ze(E)/HF azeotropes, HFO-1234yf (2,3,3,3-tetrafluoropropene), HFC-245cb (1,1,1,2,2-pentafluoropropane), HFC-152a (1,1-difluoroethane), HFC-134a (1,1,1,2-tetrafluoroethane), HFC-125 (pentafluoroethane), HFO-1225ye(Z) (1,2,3,3,3,-pentafluoropropene), HFO-1234zc (1,1,3,3,3-pentafluoropropene), or HFO-1234zf (2,3,3,3-tetrafluoropropene) and combinations of the foregoing, and is conveyed to a separation device to remove the light impurities before entering the distillation apparatus to remove CFC-113.

9. The process of claim 8, wherein the separation device comprises at least one of a batch or continuous fractional distillation column, spinning band distillation equipment, a wiped film evaporator, and combinations of the foregoing.

10. The process of claim 8, wherein the level of light impurities is reduced to 1 wt. % or less based on the total weight of the intermediate stream.

11. The process of claim 1, wherein the reactor is operated at at least one of the following conditions:
(i) a temperature of about 205° C. to 370° C.;
(ii) a pressure of about −15 to 100 psia; and
(iii) a residence time of about 1 second to 100 seconds.

12. The process of claim 1, wherein the distillation apparatus is operated at least one of the following conditions:
(i) a temperature of about 10° C. to 105° C. and
(ii) a pressure of about −15 to 150 psia

13. A process for removing CFC-113 from the HFC-245fa feed stream in the production of HFO-1234ze(E), comprising:
feeding HFC-245fa to a separation device, the HFC-245fa further comprising a first amount of CFC-113,
recovering, from the separation device, a purified HFC-245fa stream having a second amount of CFC-113 less than the first amount.

14. The process of claim 13, wherein the separation device comprises at least one of zeolites, liquid extractants, azeotropic distillation apparatuses, mineral oil, and combinations of the foregoing.

15. The process of claim 13, wherein the purified HFC-245fa stream comprises less than 0.05 wt. % CFC-113, based on a total weight of the purified HFC-245fa stream.

16. A composition comprising an azeotrope or azeotrope-like composition consisting essentially of effective amounts of CFC-113 and HFC-245fa.

17. The composition of claim 16, wherein the azeotrope or azeotrope-like composition consists essentially of about 3.5 wt. % CFC-113 and about 96.5 wt. % HFC-245fa.
